(19) United States
(12) Patent Application Publication
(10) Pub. No.: US 2013/0018274 A1
(43) Pub. Date: Jan. 17, 2013

(54) SYSTEM AND DEVICE FOR TESTING PULMONARY FUNCTION

(71) Applicant: Alfonso V. O'Neill, Grosse Pointe Farms, MI (US)

(72) Inventor: Alfonso V. O'Neill, Grosse Pointe Farms, MI (US)

(21) Appl. No.: 13/547,862

(22) Filed: Jul. 12, 2012

Related U.S. Application Data
(60) Provisional application No. 61/507,562, filed on Jul. 13, 2011.

(51) Int. Cl. A61B 5/087; A61B 5/08

(52) U.S. Cl. 600/532; 600/529; 600/538

(57) ABSTRACT

A method for analyzing lunch function includes receiving, at a signal generating device, an output of a respiratory maneuver, where the output of the respiratory maneuver includes one or more respiratory parameters. The method also includes producing, at the signal generating device, a signal corresponding to the output of the respiratory maneuver based on the one or more respiratory parameters, wherein the signal describes one or more characteristics of the respiratory maneuver. The method further comprises transmitting the signal from the signal generating device to a signal receiving device at a remote location over a network.

### CROSS-REFERENCE TO RELATED APPLICATIONS

The present application claims the benefit of U.S. Provisional Patent Application No. 61/507,562, filed on Jul. 13, 2011, which is incorporated herein by reference in its entirety.

### BACKGROUND

The present application relates generally to the field of medical devices. More specifically, the application relates to a system and device for measuring the respiratory function of a person and remotely evaluating the results of the measurement in order to assess the respiratory health of the patient.

Respiratory problems, such as chronic obstructive pulmonary disease (COPD) and asthma, are common in society. COPD is a disease which causes the airways of the lungs to narrow, thereby limiting the airflow into and out of the lungs. COPD is a leading cause of death in the United States and creates a heavy economic burden by creating high health care costs and leading to lost worker productivity. Asthma is a chronic condition of the respiratory system whereby the airways may become inflamed and constrict, which typically leads to shortness of breath, coughing and tightening of the chest, but may be fatal in certain circumstances. Asthma may be triggered by a myriad of triggers or stimulants, for example the common cold, smoke inhalation, or exercise.

It has been known to diagnose respiratory problems such as COPD with a device known as a spirometer, which includes a differential pressure transducer for measuring the volume of air that is inhaled into and expired from the lungs. It has also been known to construct a hand-held electronic spirometric device, sometimes called a peak flow meter, to measure the exhaled air flow rate and air flow temperature to determine the air flow volume. These devices allow a patient to obtain a metric on lung performance, but lack the desired element of physician analysis and diagnosis.

It would be useful to provide a person with a readily available (e.g., over-the-counter) low cost system to analyze the respiratory health of the person from the comfort of their own home (or any location) and still have a qualified physician review the results and make a determination as to the health of the person. It would also be useful for the person being analyzed to be provided timely feedback as to their current respiratory health and whether further treatment is required. Such a low cost system would be available to a larger number of people who would be provided with an early detection method of a potential serious condition to prevent hospitalization or more serious outcomes, which can occur from not seeking medical treatment of a potentially fatal health condition.

### SUMMARY

According to an exemplary embodiment, a method for analyzing lunch function includes receiving, at a signal generating device, an output of a respiratory maneuver, where the output of the respiratory maneuver includes one or more respiratory parameters. The method also includes producing, at the signal generating device, a signal corresponding to the output of the respiratory maneuver based on the one or more respiratory parameters, wherein the signal describes one or more characteristics of the respiratory maneuver. The method further comprises transmitting the signal from the signal generating device to a signal receiving device at a remote location over a network.

Another exemplary embodiment relates to a signal generating device that includes a memory configured to store computer-readable instructions and a processor configured to execute the computer-readable instructions to perform operations comprising: receiving an output of a respiratory maneuver, wherein the output of the respiratory maneuver comprises one or more respiratory parameters; producing a signal corresponding to the output of the respiratory maneuver based on the one or more respiratory parameters, wherein the signal describes one or more characteristics of the respiratory maneuver; and transmitting the signal to a signal receiving device at a remote location over a network.

Another exemplary embodiment relates to a method for analyzing lung function. The method includes receiving a signal at a signal receiving device from a remotely located signal generating device, wherein the signal describes one or more parameters of an output of a respiratory maneuver. The method also includes analyzing, at the signal receiving device, the signal to discern one or more characteristics of the respiratory maneuver.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE DRAWINGS

FIG. 1 is a flow diagram illustrating an exemplary method of evaluating a person's respiratory health in accordance with an illustrative embodiment.

FIG. 2 is a flow diagram illustrating an exemplary method of evaluating a person's respiratory health in accordance with an illustrative embodiment.

FIG. 3 is a diagram illustrating the flow of data from a signal generating device to a signal receiving device in accordance with an illustrative embodiment.

FIG. 4a is a complete flow loop diagram in accordance with an illustrative embodiment.

FIG. 4b is the expiratory portion of the flow loop diagram of FIG. 3a in accordance with an illustrative embodiment.

FIG. 4c is a graph illustrating lung volume versus time in accordance with an illustrative embodiment.

### DETAILED DESCRIPTION

The present application discloses a low cost, timely, and convenient method for a person to have their pulmonary function tested, reviewed by a physician or other reviewing entity, and then receive a determination as to their respiratory health from the reviewing physician or reviewing entity as well as professional advice whether to seek additional treatment. The present application also discloses the devices to facilitate the pulmonary function testing. The method involves a person obtaining a signal generating device which the person may use to produce an audible tone or electronic signal by executing a respiratory/expiratory maneuver such as exhaling or blowing air through or into the device. In an embodiment, the signal generating device may be a hand-held over-the-counter device.

The signal generating device would translate the respiratory parameters (e.g., volume flow rate, air pressure gradient/drop, eddy viscosity/turbulence, chemical analysis, etc.) of an output of a respiratory maneuver of the person into a corresponding sound (e.g., pitch, tone, etc.) or data signal (analog or digital), which is communicated by a communication system (e.g., telephone network, cellular network, Internet, local wireless or wired network, Bluetooth, etc.) to a predetermined location that contains a signal receiving device. The sound may be in analog, digital or any other acceptable format for transmission known to those of skill in the art. The signal receiving device (e.g., computer with translation software) receives the sound or data signal associated with the output of the respiratory maneuver of the person and recreates the required respiratory parameters (e.g., volume flow rate loop) as desired by the physician which may be reviewed by the physician to determine the respiratory health of the person. In an embodiment, the respiratory maneuver may include an expiratory maneuver (e.g., a person exhaling into the signal receiving device) or any other suitable respiratory maneuvers known to those of skill in the art. The reviewing physician may then recommend the appropriate medical care, depending upon the health status of the person. This recommendation may be made through the method (e.g., phone, email, fax) previously selected by the person having their respiratory health analyzed.

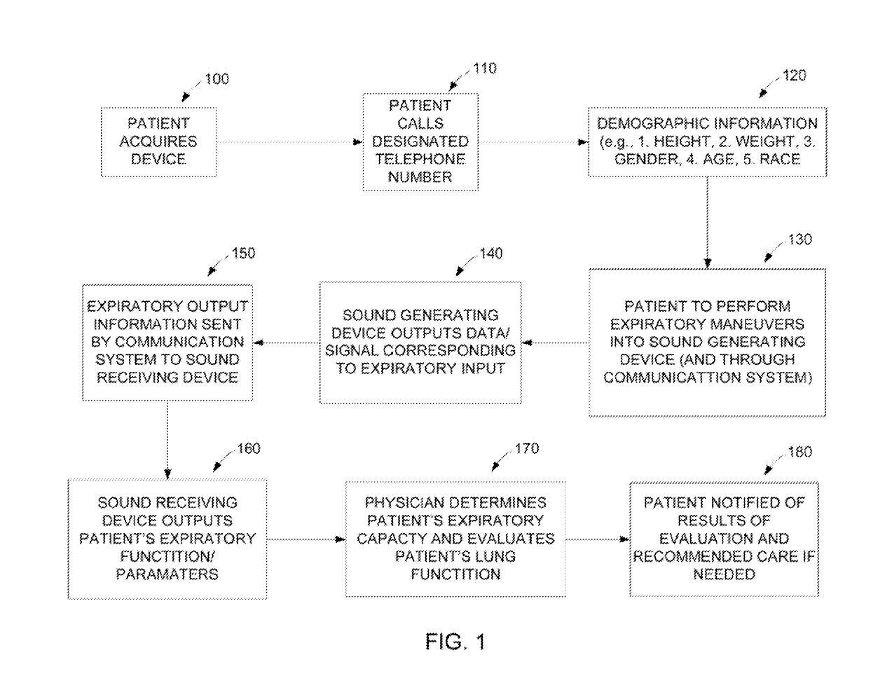

FIG. 1 is a flow diagram illustrating an exemplary method of evaluating a person's respiratory health in accordance with an illustrative embodiment. In an operation 100, a signal generating device, which may be offered over-the-counter (i.e., without the requirement to obtain a prescription from a physician or other medical professional), is acquired to enable performance of the pulmonary test process from the privacy of a person's home or from another desired location. In an operation 110, the person calls a predetermined telephone number. In an operation 120, the person follows a series of predetermined prompted questions to provide the demographic information (e.g., height, weight, age, gender, race) of the person having their pulmonary function tested and analyzed. In an operation 130, the person to be tested follows predetermined provided instructions and performs a required number (e.g., 3) of respiratory exercises or maneuvers, lasting a given duration (e.g., 6 seconds, 8 seconds, etc.), through the signal generating device and also through a communication device (e.g., telephone, cellular phone, smart phone, computer, etc.). Other methods to transmit the signal generated by the signal generating device may be used, such as a microphone coupled to a computer, wherein the transmission method is able to communicate to the signal receiving device used by a reviewing physician or entity.

The signal generating device may be hand-held, compact in geometry, and lightweight. The signal generating device is configured to receive, as an input, the output of the respiratory maneuver of the person being tested. The output of the respiratory maneuver includes any number of respiratory parameters. The respiratory parameters (e.g., flow rate measured in volume per unit time such as liters/second, air pressure gradient/drop, eddy viscosity/turbulence, chemical analysis, etc.) from the output of each respiratory maneuver provided by the person being tested may be transformed by the signal generating device into a predetermined sound (e.g., pitch, tone), whereby each sound corresponds to a specific parameter (e.g., specific flow rate, specific air pressure gradient/drop, specific eddy viscosity or turbulence, or specific chemical composition). For example, for the parameter of flow rate, the signal generating device would measure the volume flow rate for the entire respiratory maneuver from the person, and may emit as an output a predetermined sound that has unique characteristics (e.g., specific pitch) that corresponds to each predetermined flow rate (i.e., a specific volume per unit time). For example, the signal generating device may generate a sound corresponding to the rotation (e.g., revolutions per minute) of a rotating flywheel driven by a person's expiratory maneuver. According to an exemplary embodiment, the pitch or frequency of the sound created by the signal generating device may be correlated to the flow rate (or other parameter) such that a higher pitch represents a higher specific value of flow rate in liters per second (or other desired unit of measurement), and where a lower pitch represents a lower specific value of flow rate in liters per second. The pitch over the duration of each respiratory maneuver may vary with respect to time, since the specific parameter (e.g., flow rate, air pressure gradient/drop, eddy viscosity/turbulence, chemical analysis) is varying with respect to time. Therefore, the output from the signal generating device may be a continuously varying pitch over time that corresponds to a continuously varying and correlated flow rate (or other parameter) over time.

According to another embodiment, the signal generating device may generate a sound at predetermined intervals of time (e.g., each 0.5 second, each 1.0 second, etc.) which would correspond to the value (or magnitude) of the respiratory parameter being received at the corresponding time of the respiratory maneuver. For example, the signal generating device may generate a “click” at a frequency corresponding to the respiratory maneuver. In another embodiment, the signal generating device may be electromechanical and cause a switch to cycle between ON or OFF and open or closed at a frequency or interval determined by the respiratory maneuver. The interval could be at any predetermined unit of time. In still another embodiment, the signal generating device may be configured to generate a non-acoustical electrical/data signal that corresponds to the output of the respiratory maneuver. For example, the signal generating device may be configured to generate an analog or digital signal that may be transmitted over a data network or other form of communication (e.g., Bluetooth, Internet, cellular, VoIP, Radio Frequency Communication (RFQ), Near Frequency Communication (NFQ)). Thus, the signal generating device may be configured to generate either an audible sound and/or an electronic data signal corresponding to the output of the respiratory maneuver.

According to another embodiment, the signal generating device may generate an initial sound or signal at the start of the respiratory maneuver, then generate additional sounds or signals corresponding to predetermined levels of change in the parameter of the respiratory maneuver being measured. For example, for flow rate, an initial sound or signal would be generated that may correspond to a correlated volume per unit time being expired from the person and when the flow rate being expired changes by a predetermined amount, the signal generating device may emit a different sound or signal corresponding to the magnitude of the change of the flow rate. The number of sounds or signals being emitted may then be a function of the change in the specific respiratory parameter being measured relative to the predetermined change in the same parameter, over the duration of the respiratory maneuver. In another example, a change in the air pressure or eddy viscosity/turbulence associated with the respiratory maneuver may be measured and used by the signal generating device.

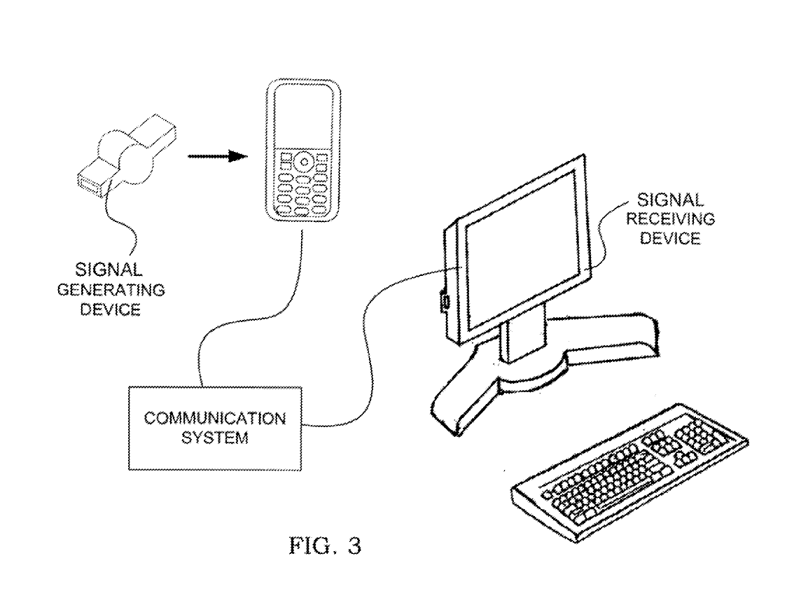

In an operation 140, the signal generating device emits the specific audible sounds or electronic signals corresponding to the specific respiratory parameters versus time, for the entire duration of each respiratory maneuver. In an operation 150, the emitted sounds may be converted to an electronic data signal and transferred to the signal receiving device through a conventional transmission method (e.g., telephone, cellular phone, computer), as shown in FIG. 3. This conversion may take the continuous analog input of the respiratory maneuver of the person and output a discrete data set to be analyzed by the signal receiving device. The conversion of the sound to an electronic format may be done according to conventional methods. In an embodiment where the signal generating device generates a specific non-acoustical electronic data signal that corresponds to the respiratory maneuver, the generating signal may not need to be converted to another electronic data signal and operation 150 may be omitted. In an embodiment, the signal receiving device is located remotely from the person being tested. The sound or signal may be transmitted over a phone network (e.g., a traditional public switched telephone network, a cellular network, etc.) or via any other network or communication method known to those of skill in the art.

In an operation 160, the signal receiving device receives the output sound or signal from the signal generating device. The signal receiving device may include a conventional computing device having software that analyzes or processes the received signal to determine the measured respiratory parameters received from the patient. For example, the signal receiving device may reconstruct the flow loop of the respiratory maneuver from the person, as either flow rate over volume or flow rate over time. According to an exemplary embodiment, the signal receiving device receives the electronic data signal that corresponds to the flow rate (or other respiratory parameter) produced by the person being analyzed, processes the series of data, and generates a continuous flow loop for the physician to evaluate to determine the health of the person being analyzed. The signal receiving device may also produce additional useful information for the reviewing physician. According to other embodiments, the signal receiving device may receive the sound data or signal data as a continuous or discrete set of data to generate the flow loop (or other depiction of data corresponding to the patient's respiratory maneuver). For discrete data sets, the signal receiving device may interpret data based on predetermined intervals of time, based on predetermined changes in parameter, or other methods to assist in a determination of the respiratory health of the person.

In an operation 170, the physician or other reviewing entity may review the flow loop or other desired output in conjunction with the demographic information provided by the person and determine the respiratory health of the person being analyzed. In an operation 180, the reviewing physician or other reviewing entity may provide the results of the evaluation of the respiratory data to the person in a manner (e.g., telephone, email, mail, etc.) chosen by the person being analyzed. Accordingly, the reviewing physician or other reviewing entity may provide a timely and remote response to the person as to their respiratory health, and if the lung function of the person is not stable, may advise as to whether to seek additional medical treatment. The reviewing physician or other entity may also recommend an appropriate location, treatment method, or any other helpful information as needed.

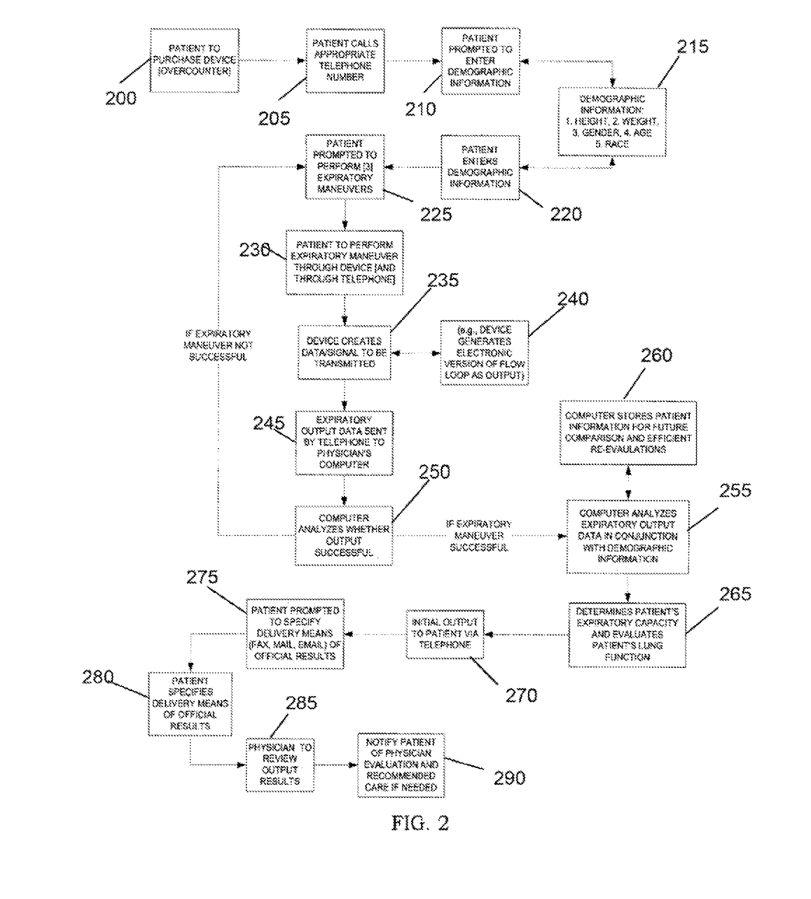

FIG. 2 is a flow diagram illustrating an exemplary method of evaluating a person's respiratory health in accordance with another illustrative embodiment. In an operation 200, a signal generating device, which may be offered over-the-counter, is acquired to enable performance of the pulmonary test process from the privacy of a person's home or from another desired location. In an operation 205, the person calls a telephone number associated with a signal receiving device that is configured to analyze information provided by the signal generating device. In an operation 210, the person is prompted to enter various information relating to demographic information of the person having their pulmonary function tested and analyzed. The demographic information may include height, weight, age, gender, race, etc. and may be displayed to the user in an operation 215. In an operation 220, the person enters the requested demographic information.

In an operation 225, the person to be tested is prompted to perform a requested respiratory maneuver. In an operation 230, the person performs the requested respiratory maneuver. In an embodiment, the requested respiratory maneuver may include an expiratory exercise and may be performed a required number of times (e.g., 3), lasting a given requested duration (e.g., 6 seconds, 8 seconds, etc.). The respiratory maneuver is performed such that an output of the respiratory maneuver is received at the signal generating device as an input. The signal generating device is further configured to generate a signal/output which may be communicated to a signal receiving device through a communication device (e.g., telephone, cellular phone, smart phone, computer, etc.). Other methods to transmit the signal generated by the signal generating device to the communication device may be used, such as a microphone coupled to a computer, wherein the transmission method is able to communicate to the signal receiving device used by a reviewing physician or entity.

In an operation 235, the signal generating device creates a data signal corresponding to the parameters of the output of the person's respiratory maneuver received during the respiratory maneuver. In an embodiment, the parameters of the output of the respiratory maneuver (e.g., flow rate measured in volume per unit time such as liters/second, air pressure gradient/drop, eddy viscosity/turbulence, chemical analysis, etc.) may be transformed by the signal generating device into a predetermined audible sound (e.g., pitch, tone), whereby different sounds correspond to a specific parameter (e.g., specific flow rate, specific air pressure gradient/drop, specific eddy viscosity or turbulence, or specific chemical composition). In another embodiment, the signal generating device may be configured to generate a non-acoustical electrical/data signal that corresponds to the respiratory maneuver. For example, in an operation 240, the signal generating device generates an electronic version of a flow loop that corresponds to the input parameters of the person's respiratory maneuver. The signal generating device may be configured to generate an analog or digital signal that may be transmitted over a data network or other form of communication (e.g., Bluetooth, Internet, cellular, PSTN, VoIP, Radio Frequency Communication (RFQ), Near Frequency Communication (NFQ)). Thus, the signal generating device may be configured to generate either an au dible sound and/or an electronic data signal corresponding to the output of the respiratory maneuver.

In an operation 245, an output signal from the signal generating device is communicated to the signal receiving device. In an embodiment, the signal receiving device is located remotely from the signal generating device and from the person being tested. In an embodiment, the output signal from the signal generating device may be received by a telephone or other communication device and transmitted over a data network or other form of communication (e.g., Bluetooth, Internet, cellular, PSTN, VoIP, Radio Frequency Communication (RFQ), Near Frequency Communication (NFQ)) to the signal receiving device. In an embodiment where the signal receiving device generates an audible sound as an output, the audible sound may be converted to an electronic data signal and transferred to the signal receiving device through a conventional transmission method (e.g., telephone, cellular phone, computer), as shown in FIG. 3. This conversion may take the continuous analog input of the respiratory maneuver of the person and output a discrete data set to be analyzed by the signal receiving device. The conversion of the sound to an electronic format may be done according to any method known to those of skill in the art, either known or later developed.

In an operation 250, the signal receiving device receives the output signal from the signal generating device and determines whether the output is successful. If the signal receiving device determines that the output is not successful, the method returns to operation 225 and the person is again prompted to perform the respiratory maneuver. If the signal receiving device determines that the output is successful, the signal receiving device analyzes the output signal in conjunction with the demographic information of the person in an operation 255. In analyzing the output signal, the signal receiving device processes the received output signal to determine the measured respiratory parameters from respiratory maneuver of the patient. For example, the signal receiving device may reconstruct the flow loop of the respiratory maneuver from the person, as either flow rate over volume or flow rate over time. According to an exemplary embodiment, the signal receiving device receives the electronic data signal that corresponds to the flow rate (or other respiratory parameter) produced by the person being analyzed, processes the series of data, and generates a continuous flow loop for the physician to evaluate to determine the health of the person being analyzed. The signal receiving device may also produce additional useful information for the reviewing physician. According to other embodiments, the signal receiving device may receive the sound data or signal data as a continuous or discrete set of data to generate the flow loop (or other depiction of data corresponding to the patient's respiratory maneuver). For discrete data sets, the signal receiving device may interpret data based on predetermined intervals of time, based on predetermined changes in parameter, or other methods to assist in a determination of the respiratory health of the person. In an operation 260, the signal receiving device stores the person's information for future comparison to subsequently received information to enable efficient and valuable historical comparisons.

In an operation 265, a physician, computing device, or other reviewing entity may review the flow loop or other information generated by the signal receiving device in conjunction with the demographic information provided by the person and evaluate the respiratory health of the person being analyzed. The evaluation may include a determination of the person's expiratory capacity and an evaluation of the person's lung function.

In an operation 270, an initial evaluation may be provided from the reviewing physician, computing device, or other reviewing entity to the person via telephone or other convenient and rapid form of communication. In an operation 275, the person may be prompted to specify a preferred delivery means (e.g., facsimile, mail, email, etc.) by which the person desires to receive the official results of the evaluation. In an operation 280, the person provides a selection of the preferred delivery means. In an operation 285, a physician or other reviewing entity reviews and approves the official results of the evaluation and okays the results for transmission to the person. In an operation 290, the official results along with a recommendation of care are provided to the person via the preferred delivery means selected by the person.

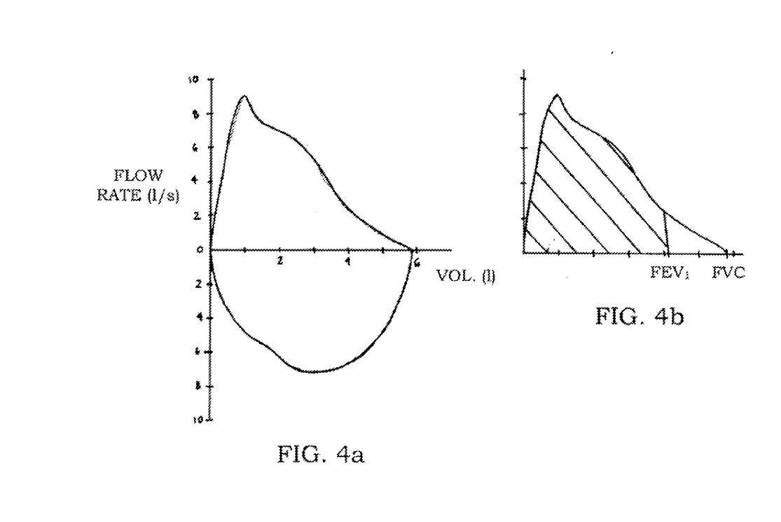

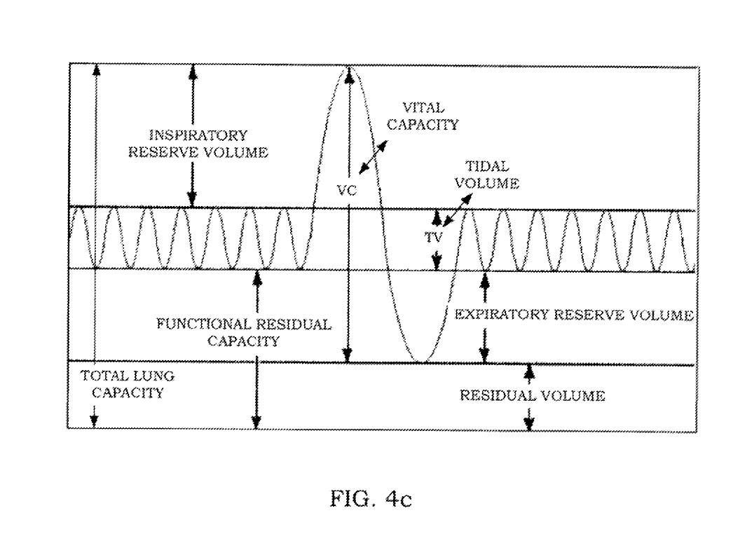

Referring to FIGS. 4a and 4b, an exemplary embodiment of a flow loop is shown. This flow loop is generated to illustrate flow rate (liters/second) versus volume (liters), whereby the top portion (above the volume axis) represents the expiratory maneuver and the lower portion (below the volume axis) represents the inspiratory maneuver. This is one method by which the signal receiving device may output the processed sound. FIG. 4b shows the expiratory portion of the flow loop and illustrates two key terms of measurement. First, is the FEV₁, which is the forced expiratory volume in one second, meaning it is the forced volume of air (here in liters) that is exhaled from the lungs during the first second of an expiratory maneuver following a full inspiratory maneuver. Second, is the FVC, which is the forced vital capacity, meaning it is the total volume of air (here in liters) that can forcibly be blown out from the lungs after a full inspiratory maneuver. FEV₁ and FVC are useful to a trained physician to determine the overall respiratory health of a patient, for example, the ratio of FEV₁/FVC may be used to detect obstructive or restrictive diseases affecting the respiratory system of the patient.

Referring to FIG. 4c, another exemplary embodiment of a flow loop is shown. This flow loop is generated to illustrate volume versus time and may be another form of output generated by the signal receiving device after receiving the data corresponding to the input sound. This flow loop is sinusoidal with the shorter amplitude peaks representing a normal breathing pattern of a person, with the single high amplitude crest representing the total lung volume following a full inspiratory maneuver. This data may be useful to a trained physician to determine the overall health of a patient.

This disclosed process being efficient and convenient allows for a person who may suffer from a chronic obstructive pulmonary disease (or any person) to have their respiratory function analyzed by a trained physician or other reviewing entity as frequently as the person desires and in a cost efficient manner. A person may have their respiratory health evaluated daily or weekly, depending on the desire of the person, from the privacy of their own home or form any convenient location. According to an exemplary embodiment, the receiving device would store the demographics of the person if desired by the person, using a personal identification method, which would make future analysis even more efficient, as the demographic information would not need to be reentered.

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the invention as recited in the appended claims.

It should be noted that the term “exemplary” or “illustrative” as used herein to describe various embodiments is intended to indicate that such embodiments are possible examples, representations, and/or illustrations of possible embodiments (and such term is not intended to connote that such embodiments are necessarily extraordinary or superlative examples).

It is important to note that the construction and arrangement of the pulmonary function system and devices as shown in the various exemplary embodiments is illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter described herein. For example, elements shown as integrally formed may be constructed of multiple parts or elements, the position of elements may be reversed or otherwise varied, and the nature or number of discrete elements or positions may be altered or varied. The order or sequence of any process or method steps may be varied or re-sequenced according to alternative embodiments. Other substitutions, modifications, changes and omissions may also be made in the design, operating conditions and arrangement of the various exemplary embodiments without departing from the scope of the present invention.

Embodiments of the subject matter and the operations described in this specification (e.g., the signal generating device, the signal receiving device, etc.) can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on one or more computer storage medium for execution by, or to control the operation of, data processing apparatus, such as a processing circuit. The signal generating and receiving devices may comprise any digital and/or analog circuit components configured to perform the functions described herein, such as a microprocessor, microcontroller, application-specific integrated circuit, programmable logic, etc. Alternatively or in addition, the program instructions can be encoded on an artificially generated propagated signal, e.g., a machine-generated electrical, optical, or electromagnetic signal, that is generated to encode information for transmission to suitable receiver apparatus for execution by a data processing apparatus.

A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate components or media (e.g., multiple CDs, disks, or other storage devices). Accordingly, the computer storage medium is both tangible and non-transitory.

The operations described in this specification can be implemented as operations performed by a data processing apparatus on data stored on one or more computer-readable storage devices or received from other sources. The term “data processing apparatus” or “computing device” encompasses all kinds of apparatus, devices, and machines for processing data, including by way of example a programmable processor, a computer, a system on a chip, or multiple ones, or combinations, of the foregoing The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

What is claimed is:

1. A method for analyzing lung function, the method comprising:
receiving, at a signal generating device, an output of a respiratory maneuver, wherein the output of the respiratory maneuver comprises one or more respiratory parameters;
producing, at the signal generating device, a signal corresponding to the output of the respiratory maneuver based on the one or more respiratory parameters, wherein the signal describes one or more characteristics of the respiratory maneuver; and
transmitting the signal from the signal generating device to a signal receiving device at a remote location over a network.

2. The method of claim 1, wherein the one or more respiratory parameters include at least one of a flow rate of the respiratory maneuver, an air pressure of the respiratory maneuver, an eddy viscosity or turbulence of the respiratory maneuver, or a specific chemical composition of the respiratory maneuver.

3. The method of claim 1, further comprising:
generating a flow loop based on the flow rate of the output of the respiratory maneuver; and
transmitting the flow loop to the signal generating device at the remote location.

4. The method of claim 1, wherein the signal corresponding to the output of the respiratory maneuver is based on one or more changes in the flow rate or air pressure of the output of the respiratory maneuver during a duration of the respiratory maneuver.

5. The method of claim 1, wherein the signal comprises an audible signal.

6. The method of claim 5, wherein at least one of a tone or a pitch of the audible signal are varied based on the one or more respiratory parameters of the output of the respiratory maneuver.

7. The method of claim 1, wherein the signal comprises an electrical data signal, the method further comprising transmitting the electrical data signal from the signal generating device to a signal receiving device, wherein the signal receiving device is configured to analyze the electrical data signal to discern the one or more characteristics of the output of the respiratory maneuver.

8. The method of claim 1, wherein the signal generating device comprises a hand-held device, and wherein the transmitting the signal from the signal generating device to the signal receiving device comprises transmitting the signal over a phone network.

9. A signal generating device comprising:
a memory configured to store computer-readable instructions; and
a processor configured to execute the computer-readable instructions to perform operations comprising:
receiving an output of a respiratory maneuver, wherein the output of the respiratory maneuver comprises one or more respiratory parameters;
producing a signal corresponding to the output of the respiratory maneuver based on the one or more respiratory parameters, wherein the signal describes one or more characteristics of the respiratory maneuver; and
transmitting the signal to a signal receiving device at a remote location over a network.

10. The signal generating device of claim 9, wherein the one or more respiratory parameters include at least one of a flow rate of the output of the respiratory maneuver, an air pressure of the output of the respiratory maneuver, an eddy viscosity or turbulence of the output of the respiratory maneuver, or a specific chemical composition of the output of the respiratory maneuver.

11. The signal generating device of claim 9, wherein the processor is configured to execute the computer-readable instructions to perform operations further comprising generating a flow loop based on the flow rate of the output of the respiratory maneuver.

12. The signal generating device of claim 9, wherein the signal corresponding to the output of the respiratory maneuver is based on one or more changes in the flow rate or air pressure of the output of the respiratory maneuver over a duration of the respiratory maneuver.

13. The signal generating device of claim 9, wherein the signal comprises an audible signal.

14. The signal generating device of claim 13, wherein at least one of a tone or a pitch of the audible signal are varied based on the one or more respiratory parameters of the output of the respiratory maneuver.

15. The signal generating device of claim 9, wherein the signal comprises an electrical data signal, and wherein the processor is configured to execute the computer-readable instructions to perform operations further comprising transmitting the electrical data signal from the signal generating device to a signal receiving device, wherein the signal receiving device is configured to analyze the electrical data signal to discern the one or more characteristics of the respiratory maneuver.

16. The signal generating device of claim 9, wherein the signal generating device comprises a hand-held device, and wherein the transmitting the signal from the signal generating device to the signal receiving device comprises transmitting the signal over a phone network.

17. A method for analyzing lung function, the method comprising:
receiving a signal at a signal receiving device from a remotely located signal generating device, wherein the signal describes one or more parameters of an output of a respiratory maneuver; and
analyzing, at the signal receiving device, the signal to discern one or more characteristics of the respiratory maneuver.

18. The method of claim 17, further comprising generating a flow loop based on the one or more parameters of the output of the respiratory maneuver.

19. The method of claim 17, wherein the signal corresponding to the respiratory maneuver is based on a change in the flow rate or air pressure of the output of the respiratory maneuver.

20. The method of claim 17, wherein the signal comprises an audible signal.

21. The method of claim 20, wherein at least one of a tone or a pitch of the audible signal are varied based on one or more respiratory parameters of the output of the respiratory maneuver.

22. The method of claim 17, further comprising providing an evaluation of lung function based on the one or more characteristics of the respiratory maneuver discerned at the signal receiving device.
